(19) United States
(12) Patent Application Publication
(10) Pub. No.: US 2004/0137424 A1
(43) Pub. Date: Jul. 15, 2004

(54) Nucleic acids and methods for detecting viral infection, uncovering anti-viral drug candidates and determining drug resistance of viral isolates

(76) Inventors: Yin Hwee Tan, St Vancouver (CA); Siew Pheng Lim, Singapore (SG); Seng Gee Lim, Singapore (SG)

Correspondence Address: WILDMAN, HARROLD, ALLEN & DIXON, 225 WEST WACKER DRIVE, CHICAGO, IL, 60606, US

(21) Appl. No.: 10/333,449

(22) PCT Filed: Jul. 20, 2001
(86) PCT No.: PCT/IL01/00669

(51) Int. Cl. C12Q 1/70; C12Q 1/68; C07H 21/02

(52) U.S. Cl. 435/5; 435/6; 536/23.72

(57) ABSTRACT

A nucleic acid construct is provided. The nucleic acid construct includes an expression cassette including: a first polynucleotide region including a 5′ NCR sequence of an RNA virus and at least an N-terminal portion of a core sequence of the RNA virus; a second polynucleotide region including a 3′ UTR sequence of the RNA virus and at least a C-terminal portion of a polymerase sequence of the virus; and a third polynucleotide region encoding a reporter molecule, the third polynucleotide region being flanked by the first and the second polynucleotide regions; and a promoter sequence being operatively linked to the expression cassette in a manner so as to enable a transcription of a minus strand RNA molecule from the expression cassette.

### FIELD AND BACKGROUND OF THE INVENTION

The present invention relates to nucleic acid constructs and methods of utilizing same for detecting infection of an RNA virus, for uncovering anti-viral drug candidates and for determining drug resistance of isolates of an RNA virus. More particularly, the present invention relates to a nucleic acid construct which transcribes a minus strand RNA sequence encoding a reporter polypeptide and including 5′ and 3′ sequences of an RNA virus. When transcribed in a cell infected with an RNA virus capable of replicating the minus strand RNA sequence, a plus strand of this RNA sequence is formed and translated by the host cell into an active reporter polypeptide.

Viral diseases are some of the major scourges of mankind and include such virulent disorders as smallpox, yellow fever, rabies, poliomyelitis and AIDS. In addition, viruses carrying oncogenes are responsible for a number of human tumors and cancers.

It is a remarkable and proven fact that some virus infections occur without overt symptoms, while others can cause more than one clinical manifestation involving more than one organ system of the body. This lack of a defining clinical manifestation in some infections, presents a major hurdle to an accurate and timely diagnosis of infections, which in some cases is crucial for the prevention of disease and death.

Several diagnostic procedures have been developed in efforts to improve the detection and diagnosis of viral infections. These procedures involve the detection of viral components in cells of infected individuals or the detection of blood components generated as a response to the presence of a viral infection. Although such methods provide acceptable accuracy in detecting some viral infections, they are oftentimes expensive and time consuming to carry out.

Although accurate and timely diagnosis of some viral infections provides clinicians with better chances of combating viral infection, the lack of suitable anti-viral drugs limits the possibilities of treatment for such viral infections

As such, for the past decades, universities and pharmaceutical companies have invested considerable resources in efforts to uncover potential anti-viral drug candidates and/or to determine the anti-viral drug resistance of some viruses.

Present day anti-viral drug screening methods rely on detecting interactions between viral components and molecules having potential anti-viral activity. For example, the identification of inhibitors of virally encoded proteases (“protease inhibitors”) relies on the in-vitro screening of purified viral protease with chemical compounds in the presence of synthetic peptide substrates. Initial in-vitro screening is usually followed by a bioassay designed for determining whether a potential protease inhibitor or its derivatives function in virally infected cells prior to additional testing conducted in more complex biological systems.

Screening for drug resistance of certain virus isolates is typically effected by phenotypic testing (plaque reduction assay). This is a labor intensive, time consuming and expensive technique that oftentimes does not correlate well to the clinical response to drug therapy in individual patients. Nonetheless, because of its derivation from testing for sensitivity to antibacterial agents, this technique is often considered to be the “gold standard”.

Prior art drug and drug resistance screening methods, such as the methods described above, are further limited in that such methods are not readily utilizable in screening for molecules possessing anti-viral activities against, nor can they be utilized to determine the drug resistance of, RNA viruses.

A large portion of the viruses responsible for human diseases are RNA viruses. Since the RNA genome of such viruses is replicated via an RNA intermediate, recombinant manipulation thereof for the purposes of constructing cell, or cell free assays is oftentimes a difficult task. In addition, the high heterogeneity of RNA viral genomes further complicates recombinant manipulation and also limits the accuracy of prior art cell free drug and drug resistance screenings.

One example of a disease causing RNA virus is the Hepatitis C virus (HCV) which is a member of the Flayiviridae family, and the major cause of chronic liver disease worldwide (1, 2). HCV is an enveloped virus with a single-stranded, positive sense, RNA genome that encodes a single open reading frame (ORF) of about 3010 amino acids (aa) which is co-translationally and post-translationally cleaved to give rise to at least 10 polypeptides (3). Located at its N-terminal end are three structural proteins, followed by at least seven non-structural (NS) proteins (1). Combined action of host-derived signal peptidase(s) and the virus-encoded proteases are involved in the processing of this polyprotein (4-8).

Similar to other RNA viruses, the genome of HCV is highly heterogeneous, and several genotypes and subtypes have been described (12, 13). Numerous studies have successfully demonstrated partial replication of the virus in in-vitro culture systems using human T-cells, B-cells (9, 10), human hepatocytes (11, 12) or chimpanzee hepatocytes (13, 14). However, these systems suffer from low viral replication efficiency and limited passage cycles. More recently, high level replication of subgenomic HCV RNA was established in a human hepatoma cell line that would enable long-term production of viral RNA and proteins (14). Unfortunately, the complete life cycle of virus does not take place in this system nor are injectable virions produced as transfection with the full length genome failed to produce any viable cell clones (14).

Replication of HCV in vivo involves the replication of its single positive-stranded RNA through negative (anti-sense) strand intermediates via the NS5B polymerase (15-17). The negative strand RNA formed then serves as a template for the synthesis of more positive RNA strands which are either used as templates for translation of viral proteins or packaged for production of viral particles. Binding and initiation of reverse strand synthesis by NS5B is dependent on stem-loop structures present in the 3′ of the viral genome (17, 18). Based on this knowledge the inventors of the present invention decided to create a reporter system using constructs encoding anti-sense luciferase gene flanked by HCV 5′ and 3′ NCR.

While reducing the present invention to practice, a cDNA clone encoding a complete HCV genome was generated by the present inventors. Sequences derived from this cDNA clone were incorporated in novel chimeric HCV-luciferase expression constructs which can be used, according to the teachings of the present invention, in accurate and rapid cell based assays for detecting HCV infection, screening molecules for potential anti-viral activities and determining drug resistance of HCV isolates.

### SUMMARY OF THE INVENTION

According to one aspect of the present invention there is provided a nucleic acid construct comprising: (a) an expression cassette including: (i) a first polynucleotide region including a 5′ NCR sequence of an RNA virus and at least an N-terminal portion of a coding sequence of the RNA virus; (ii) a second polynucleotide region including a 3′ UTR sequence of the RNA virus and at least a C-terminal portion of a coding sequence of the virus; and (iii) a third polynucleotide region encoding a reporter molecule, the third polynucleotide region being flanked by the first and the second polynucleotide regions; and (b) a promoter sequence being operatively linked to the expression cassette in a manner so as to enable a transcription of a minus strand RNA molecule from the expression cassette.

According to another aspect of the present invention there is provided a genetically transformed cell comprising a nucleic acid construct including: (a) an expression cassette including: (i) a first polynucleotide region including a 5′ NCR sequence of an RNA virus and at least an N-terminal portion of a coding sequence of the RNA virus; (ii) a second polynucleotide region including a 3′ UTR sequence of the RNA virus and at least a C-terminal portion of a coding sequence of the virus; and (iii) a third polynucleotide region encoding a reporter molecule, the third polynucleotide region being flanked by the first and the second polynucleotide regions; and (b) a promoter sequence being operatively linked to the expression cassette in a manner so as to enable a transcription of a minus strand RNA molecule from the expression cassette.

According to further features in preferred embodiments of the invention described below, the genetically transformed cell further comprising an additional nucleic acid construct for expressing at least an RNA dependent RNA polymerase of a virus, whereas the first and the second polynucleotide regions being selected such that the RNA dependent RNA polymerase is capable of replicating the minus strand RNA molecule into plus strand RNA.

According to still further features in the described preferred embodiments at least a portion of the first polynucleotide region is at least 50% identical to a sequence encompassed by nucleotides 1-374 of SEQ ID NO:33.

According to still further features in the described preferred embodiments at least a portion of the second polynucleotide region is at least 50% identical to a sequence encompassed by nucleotides 9158-9609 of SEQ ID NO:33.

According to still further features in the described preferred embodiments the first polynucleotide region further includes a 5′ UTR sequence of the RNA virus.

According to still further features in the described preferred embodiments the first polynucleotide region includes an IRES sequence.

According to still further features in the described preferred embodiments the RNA virus is selected from the group consisting of a positive strand RNA virus and a negative strand RNA virus.

According to still further features in the described preferred embodiments the RNA virus is selected from the group consisting of a virus of the picomavirus family, a virus of the togavirus family, a virus of the orthomyxovirus family, a virus of the paramyxovirus family, a virus of the coronavirus family, a virus of the calicivirus family, a virus of the arenavirus family, a virus of the rhabdovirus family and a virus of the bunyavirus family.

According to still further features in the described preferred embodiments the RNA virus is Hepatitis C.

According to still further features in the described preferred embodiments the first and the second polynucleotide regions are selected such that the minus strand RNA molecule transcribable from the expression cassette is replicatable by an RNA dependent RNA polymerase of the virus into a plus strand RNA molecule.

According to still further features in the described preferred embodiments the promoter is functional in a eukaryotic cell.

According to still further features in the described preferred embodiments the eukaryotic cell is selected from the group consisting of an insect cell, a yeast cell and a mammalian cell.

According to still further features in the described preferred embodiments the reporter molecule is a polypeptide selected from the group consisting of an enzyme, a fluorophore, a substrate and a ligand.

According to yet another aspect of the present invention there is provided a method of detecting a presence of an RNA virus in a cell, the method comprising the steps of: (a) incubating a nucleic acid construct with an extract of the cell under conditions suitable for transcription and translation of the nucleic acid construct, the nucleic acid construct including: (i) an expression cassette having: (one) a first polynucleotide region including a 5′ NCR sequence of an RNA virus and at least an N-terminal portion of a coding sequence of the RNA virus; (two) a second polynucleotide region including a 3′ UTR sequence of the RNA virus and at least a C-terminal portion of a coding sequence of the virus; and (three) a third polynucleotide region encoding a reporter molecule, the third polynucleotide region being flanked by the first and the second polynucleotide regions; and (ii) a promoter sequence being operatively linked to the expression cassette in a manner so as to direct the transcription of a minus strand RNA molecule from the expression cassette when the nucleic acid construct is incubated with the extract, the first and the second polynucleotide regions being selected such that the minus strand RNA molecule transcribed is replicatable by the polymerase of the RNA virus into a plus strand RNA molecule; and (b) quantifying a level of the reporter molecule to thereby determine the presence of the virus in the cell.

According to still further features in the described preferred embodiments the reporter molecule is a polypeptide translated from the plus strand RNA molecule.

According to still further features in the described preferred embodiments the method described above further comprising the step of comparing the level of the reporter molecule to that obtained from cells free of the virus.

According to a further aspect of the present invention there is provided a method of screening for anti-viral drugs, the method comprising the steps of: (a) co-incubating a nucleic acid construct, a polynucleotide encoding at least a polymerase of an RNA virus and a potential anti-viral molecule under conditions suitable for transcription and translation of the nucleic acid construct and the polynucleotide encoding at least the polymerase, the nucleic acid construct including: (i) an expression cassette having: (one) a first polynucleotide region including a 5′ NCR sequence of an RNA virus and at least an N-terminal portion of a coding sequence of the RNA virus; (two) a second polynucleotide region including a 3′ UTR sequence of the RNA virus and at least a C-terminal portion of a coding sequence of the virus; and

(three) a third polynucleotide region encoding a reporter molecule, the third polynucleotide region being flanked by the first and the second polynucleotide regions; and (ii) a promoter sequence being operatively linked to the expression cassette in a manner so as to direct the transcription of a minus strand RNA molecule from the expression cassette when the nucleic acid construct is incubated with the polynucleotide encoding the polymerase of the RNA virus under the conditions suitable for transcription and translation, the first and the second polynucleotide regions being selected such that the minus strand RNA molecule transcribed is replicatable by the polymerase of the RNA virus into a plus strand RNA molecule; and (b) quantifying a level of the reporter molecule to thereby determine the anti-viral activity of the potential anti-viral molecule.

According to still further features in the described preferred embodiments the reporter molecule is a polypeptide translated from the plus strand RNA molecule.

According to still further features in the described preferred embodiments the method described above further comprising the step of comparing the level of the reporter molecule to that obtained from cells free of the virus.

According to still further features in the described preferred embodiments the potential anti-viral molecule is selected from the group consisting of a nucleoside or nucleotide analogue and an immune-modulatory molecule.

According to still further features in the described preferred embodiments step (a) is effected by introducing the nucleic acid construct, the polynucleotide encoding at least the polymerase of the RNA virus and the potential anti-viral molecule into a cell.

According to still further features in the described preferred embodiments step (a) is effected by introducing the nucleic acid construct and the potential anti-viral molecule into a cell infected with the RNA virus.

According to yet a further aspect of the present invention there is provided a method of determining drug resistance of an RNA virus, the method comprising the steps of: (a) co-incubating a nucleic acid construct, a polynucleotide encoding at least a polymerase of the RNA virus and an anti-viral drug molecule under conditions suitable for transcription and translation of the nucleic acid construct and the polynucleotide encoding at least the polymerase, the nucleic acid construct including: (i) an expression cassette having: (one) a first polynucleotide region including a 5′ NCR sequence of an RNA virus and at least an N-terminal portion of a coding sequence of the RNA virus; (two) a second polynucleotide region including a 3′ UTR sequence of the RNA virus and at least a C-terminal portion of a coding sequence of the virus; and (three) a third polynucleotide region encoding a reporter molecule, the third polynucleotide region being flanked by the first and the second polynucleotide regions; and (ii) a promoter sequence being operatively linked to the expression cassette in a manner so as to direct the transcription of a minus strand RNA molecule from the expression cassette when the nucleic acid construct is incubated with the polynucleotide encoding at least the polymerase of the RNA virus under the conditions suitable for transcription and translation, the first and the second polynucleotide regions being selected such that the minus strand RNA molecule transcribed is replicatable by the polymerase of the RNA virus into a plus strand RNA molecule; and (b) quantifying a level of the reporter molecule to thereby determine the resistance of the RNA virus to the anti-viral drug.

According to still further features in the described preferred embodiments the method described above further comprising the step of comparing the level of the reporter molecule to that obtained from cells free of the anti-viral drug.

According to still further features in the described preferred embodiments the reporter molecule is a polypeptide translated from the plus strand RNA molecule.

According to still further features in the described preferred embodiments the anti-viral drug is selected from the group consisting of a nucleoside or nucleotide analogue and an immune-modulatory molecule.

According to still further features in the described preferred embodiments step (a) is effected by introducing the nucleic acid construct, the polynucleotide encoding at least the polymerase of the RNA virus and the anti-viral drug into a cell.

According to still further features in the described preferred embodiments step (a) is effected by introducing the nucleic acid construct and the anti-viral drug into a cell infected with the RNA virus.

The present invention successfully addresses the shortcomings of the presently known configurations by providing nucleic acid constructs and methods of utilizing same for detecting the presence of an RNA virus in a cell or a cell extract, for uncovering novel anti-viral drugs and for determining the resistance of RNA virus isolates to anti-viral drugs.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a schematic representation of the overlapping HCV cDNA clones of HCV-S1 utilized in constructing the HCV genome. The positions of the first and last nucleotides and amino acids of the individual HCV proteins as well as the first and last nucleotide of the HCV 5′ UTR and 3′ UTR are indicated. Clones A-M represent the overlapping cDNA clones of HCV-S1 obtained from RT-PCR. The first and last nucleotide of each clone is indicated.

FIG. 1B illustrates the step employed for constructing the sense and antisense chimeric vectors of the present invention.

FIGS. 2A-C illustrate the protein products of in vitro translation experiments of HCV constructs separated on SDS-PAGE. FIG. 2A—translation of the entire non-structural HCV polyprotein from pcDNA3(NSP). FIG. 2B—translation of the entire structural HCV polyprotein from pcDNA(SP). FIG. 2C—translation of the full length HCV genome from pcDNA3(S1). CPMM represents incubation with canine pancreatic microsomal membranes. Arrows indicate positions of autolytically cleaved products upon prolonged incubation. Molecular weight marker sizes (in kDa) are indicated on the left.

FIGS. 3A-G illustrate western analysis of 293T cells transiently transfected with pXJ41(S1). Cells were harvested two days post-transfection and lysate proteins were separated on SDS-PAGE gels and transferred onto nitrocellulose membranes. The blotted proteins were probed with anti-E2 (FIG. 3A), anti-NS3 (FIG. 3B) and anti-NS5A (FIG. 3C) monoclonal antibodies. The detection of core (FIGS. 3E-G) and NS5B (FIGS. 3D-E) proteins, was effected using different sera from HCV infected patient at a dilution of 1:100 (FIGS. 3D-G). The immunoblots of FIGS. 3D-E represent sera taken from the same patient from which the HCV-S1 was cloned. Molecular weight marker sizes (in kDa) are indicated on the left.

### DESCRIPTION OF THE PREFERRED EMBODIMENTS

The present invention is of nucleic acid constructs and methods utilizing same which can be utilized for detecting infection of an RNA virus, for uncovering anti-viral drug candidates and for determining drug resistance of isolates of an RNA virus. Specifically, the present invention is of a nucleic acid construct which transcribes a minus strand RNA sequence encoding a reporter polypeptide and including 5′ and 3′ sequences of an RNA virus. When transcribed in a cell infected with an RNA virus capable of replicating the minus strand RNA sequence, a plus strand of this RNA sequence is formed and translated by the host cell into an active reporter polypeptide.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The molecular studies of the pathogenesis of HCV and the development of anti-viral drugs have been hampered in part by the lack of a robust, cell-based assay to monitor viral replication. The currently available cell-based systems are limited by the low viral replication efficiency and limited passage cycles. Although high levels of replication of subgenomic HCV RNA was established in a human hepatoma cell line that would enable long-term production of viral RNA and proteins, this does not truly measure viral replication. The complete life cycle of HCV does not occur in this system, nor are injectable virions produced. Moreover, the authors failed to generate any viable cell clones when they carried out transfections with the full length genome (14).

Replication of the HCV genome in vivo is dependent in part on the proteolytic activity of host signal peptidase(s) for cleavage of its structural genes and on its NS3 protein, which systematically cleaves the viral NS polyprotein to release the individual active subunits (7). Of these, the viral RNA dependent RNA polymerase, NS5B, plays a vital role in replication through synthesis of both positive and negative viral RNA strands (15). Due to the low replication efficiency of HCV, nested RT-PCR for amplifying minus-strand RNA is employed to determine viral replication in vivo. This method is both laborious and easily prone to false positive errors. Although its sensitivity and reliability has been improved with the use of tagged primers and Tth polymerase (13), it still remains expensive and time-consuming.

As is further described in the Examples section which follows, to generate a reliable and simple reporter assay system which can be utilized to detect hepatitis C virus (HCV) replication in vivo, and to uncover novel anti-viral drugs as well as to screen for drug resistance in viral isolates, the present inventors undertook the laborious task of generating a replication-competent full length HCV genome.

Sequences derived from this clone were then utilized to generate reporter expression constructs which produce a reporter signal in the presence of infecting virus particles.

Thus, according to one aspect of the present invention there is provided a nucleic acid construct. The nucleic acid construct includes an expression cassette having a first polynucleotide region including a 5′ NCR sequence of an RNA virus and at least an N-terminal portion of a coding sequence of the RNA virus, such as for example the N-terminal portion of the core sequence, and a second polynucleotide region including a 3′ UTR sequence of the RNA virus and at least a C-terminal portion of a coding sequence of the virus, such as for example a C-terminal portion of the viral polymerase sequence. The expression cassette also includes a third polynucleotide region which encodes a reporter polypeptide such as for example, an enzyme, a substrate, a ligand or receptor or a fluorophore.

According to the present invention, the reporter molecule encoding region is flanked by the first and the second polynucleotide regions and is in transcriptional linkage therewith.

The nucleic acid construct according to this aspect of the present invention, also includes a promoter sequence which serves to direct transcription of the expression cassette sequence in eukaryotic cells such as for example, mammalian cells, yeast cells or insect cells.

The promoter sequence is oriented with respect to the expression cassette sequence, such that transcription therefrom generates a minus strand RNA molecule.

As used herein the phrase “minus (or negative) strand RNA” refers to the complementary RNA strand of the “plus (or positive) strand RNA” which is the strand typically translated by the ribosomes into a polypeptide sequence.

According to a preferred embodiment of the present invention, at least a portion of the first polynucleotide region is at least 50%, at least 60%, at least 70% at least 80%, at least 90to 95% identical to a sequence encompassed by nucleotides 1-374 of SEQ ID NO:33.

According to another preferred embodiment of the present invention, at least a portion of the second polynucleotide region is at least 50%, at least 60%, at least 70% at least 80%, at least 90 to 95% identical to a sequence encompassed by nucleotides 9158-9609 of SEQ ID NO:33.

Since the nucleic acid construct of the present invention transcribes a minus strand RNA molecule in cells, such a construct cannot generate an active reporter molecule in cells transformed with this construct. However, in the presence of a viral polymerase, such as the RNA dependent RNA polymerase encoded by RNA viruses (hereinafter RNA polymerase), such as the case when the transformed cell is infected with a virus or expresses the viral polymerase, replication of the minus strand RNA takes place and a plus strand RNA molecule is formed. This molecule can then be translated by the host cell ribosome into an active reporter molecule. It will be appreciated that this is true only in cases where the viral RNA polymerase binds and initiates replication from the viral sequences included within the transcribed minus strand RNA. In most cases, the viral sequences utilized in the expression cassette of the nucleic acid construct will be derived from the virus of interest, although in some cases, RNA polymerases of one virus can replicate RNA which includes 5′ and 3′ sequences from another virus.

Since the sequences regulating RNA replication in RNA viruses reside in the 5′ and 3′ NCRs and/or UTRs, such sequences alone are often sufficient in promoting RNA replication of the minus strand RNA transcribed from the nucleic acid construct of the present invention. However, not withstanding from the above, in some RNA viruses, coding region sequences are often necessary in order to initiate or enhance replication, as is the case for HCV. As such, the expression cassette according to the present invention preferably also includes such sequences, the identity thereof can be determined by quantifying replication from various expression cassettes which include different segments from the coding region of the virus.

Since the cap dependent translation of RNA in virally infected cells is oftentimes downregulated by the presence of a replicating virus, the expression cassette preferably also include internal ribosome entry site (IRES) sequences for initiation of cap independent translation of the chimeric polypeptide(s)if such sequences are not already included within the 5′ and 3′ sequences.

The viral sequences included in the expression cassette according to the present invention, are derived from a plus strand RNA virus or a minus strand RNA virus such as for example a virus of the picomavirus family, a virus of the togavirus family, a virus of the orthomyxovirus family, a virus of the paramyxovirus family, a virus of the coronavirus family, a virus of the calicivirus family, a virus of the arenavirus family, a virus of the rhabdovirus family or a virus of the bunyavirus family.

According to another preferred embodiments of the present invention, the RNA virus is a Hepatitis C virus (HCV).

The nucleic acid construct described hereinabove can be constructed using commercially available mammalian expression vectors or derivatives thereof. Examples of suitable vectors include, but are not limited to, pcDNA3, pcDNA3.1 (±), pZeoSV2(±), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives and modificants.

Any of the promoter and/or regulatory sequences included in the mammalian expression vectors described above can be utilized to direct the transcription of the expression cassettes described above. However, since such vectors are readily amenable to sequence modifications via standard recombinant techniques, additional regulatory elements, promoter and/or selection markers can easily be incorporated therein if needed.

The nucleic acid construct according to this aspect of the present invention can be utilized in a cell-based or a cell free assay to detect virus infection of a cell, to uncover novel anti-viral drugs or to determine the resistance of an RNA virus isolate to anti-viral drugs.

When utilized in cell-based assays, the nucleic acid construct is introduced into a cell via any standard transformation method. Numerous methods are known in the art for introducing exogenous polynucleotide sequences into eukaryotic cells. Such methods include, but are not limited to, direct polynucleotide uptake techniques, and virus or liposome mediated transformation (for further detail see, for example, “Methods in Enzymology” Vol. 1-317, Academic Press). Bombardment of cells or cell cultures.

A genetically transformed cell including the nucleic acid construct of the present invention either stability integrated into it's genome, or transiently expressed can be utilized for a cell-based assay. In assays designed for uncovering novel anti-viral drugs or determining the resistance of an RNA virus isolate to anti-viral drugs, such a cell can further be genetically transformed to also express an RNA polymerase of a virus of interest along with other viral proteins and as such serve as a “test bed” for various molecules of interest.

Thus, the nucleic acid construct of the present invention can be utilized in a method for detecting a presence of an RNA virus in a cell by incubating the nucleic acid construct with an extract of cell or by introducing the construct into the cell and measuring the signal from the reporter molecule. Preferably, this signal is compared to a signal measured from a cell infected with a virus and possibly also a cell not infected with the virus to thereby determine the presence of the virus in the cell.

As mentioned hereinabove, the nucleic acid construct of the present invention can be utilized in an assay designed for screening anti-viral activities of various molecules or in an assay for determining the drug resistance of an RNA virus isolate. Such assays are separately effected by incubating the nucleic acid construct and a potential anti-viral drug when screening molecules for anti-viral activities, or a known anti-viral drug when determining drug resistance of an RNA virus along with a cellular extract from an infected cell. Alternatively the constructs and potential or known drug are introduced into an infected cell or a cell expressing the viral polymerase and possibly other viral components.

Following a predetermined time period, the reporter activities are measured and preferably compared to those measured from cells not including the potential or known drug to thereby determine the anti-viral activity of the drug candidate or to determine the resistance of the virus to the known anti-viral drug.

It will be appreciated that although cell-free assays (in-vitro) can be efficiently utilized for determining the anti-viral activity of a drug candidate or for determining the resistance of the virus to the known anti-viral drug cell-based assays (in-situ) screening in virally infected cells is preferred since this method determines anti-viral activity in-situ and in the presence of all the virally expressed components and as such it is more accurate in predicting future activity of screened molecules in-vivo.

Thus, the present invention provides nucleic acid constructs and methods of utilizing same to detect viruses in infected cells, to screen and uncover potential anti-viral drugs and to determine drug resistance of virus isolates.

The present invention presents several advantages over prior art methods. It is easily to implementable and executable, and in addition when utilized for uncovering potential viral drugs and for drug resistance screening it can provide results of an accuracy which far exceeds that achieved by presently available in-vitro methods.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Clinical Characteristics of the Recipient Patient:

Sera from an individual known to be suffering both thalessemia and chronic hepatitis C was used for RT-PCR to obtain overlapping clones comprising the full length HCV genome (SEQ ID NO:33). Sera was collected from the patient after bone marrow transplantation upon diagnosis of elevated levels of serum transaminase indicative of HCV reactivation. The patient was determined to be HCV positive by RT-PCR of the plasma using the branched DNA assay with a level of 35.2 Meq/ml (Quantiplex HCV RNA assay, version 2.0 (bDNA); Chiron Diagnostics). Serum samples were collected in 400 μl aliquots and stored at −80° C.

Isolation of HCV RNA:

RNA was extracted from 400 μl of sera using 1.2 ml of the Trizol LS reagent (Gibco BRL, Gaithesburg Md., USA). The mixture was inverted for 20 seconds at room temperature (RT), 0.35 ml of chloroform was added and the mixture inverted again for 20 seconds. The mixture was allowed to stand at RT for 5 minutes following which it was centrifuged at 12 000 rpm for 20 minutes. The upper phase of the mixture was transferred to a new microfuge tube, 0.8 ml of isopropanol and mixing was effected via inversion. The tube was left at RT for 5 minutes following which it was spun again at 12 000 rpm for 20 minutes at 4° C. The RNA pellet was air-dried and re-suspended in 50 ml of DEPC-treated water.

RT-PCR:

Several RT-PCR reactions were conducted in order to obtain the various overlapping cDNA fragments. The various RT-PCR utilized are listed in Table 1. The RNA, extracted as described above, was reverse transcribed at 42° C. for 1 hour using 100 ng of oligo(dT) and/or specific antisense primers and 200 U of Superscript II polymerase (Gibco BRL, Gaithersburg). The resultant cDNA samples were heated at 70° C. for 15 minutes and PCR amplified using the Expand High Fidelity PCR System (Boehringer Mannheim). The PCR reactions were performed with 2-5 μl of template in a total volume of 50 μl. Different cycling profiles were used depending on the target length and the melting temperature (Tm) of the primers. Generally the PCR conditions were as follows: a hot-start at 95° C. for 3 min, denaturation at 95° C. for 1 min, annealing at 45-65° C. for 1 min, and extension at 68° C. for 1 min per 1 kb of amplified cDNA. At the end of 30-35 cycles, a final extension was carried out at 68° C. for 8 minutes. In several cases nested PCR was carried out to obtain the HCV cDNA fragment (Table 1).

5′ Race:

To clone the 5′ UTR of HCV, a 5′ rapid amplification of cDNA ends method using the 5′/3′ RACE kit from Boehringer Mannheim was employed. The first strand cDNA was synthesized with the antisense primer H3 (Table 1) and AM reverse transcriptase at 55° C. for 1 hour and the resultant cDNA was purified using the High Pure PCR Product Purification kit (Boehringer Mannheim). A terminal transferase was utilized for 3′ dA-tailing of the purified cDNA sample following which the transferase was heat-inactivated at 70° C. for 10 minutes. The tailed cDNA was amplified using the oligo dT-anchor primer and the H29 and the gene specific H4 primers (Table 1) utilizing the Expand High Fidelity PCR system. PCR conditions were as follows: 95° C. for 3 minutes, followed by 35 cycles of 95° C. for 1 minute, 45° C. for 1 minute, 68° C. for 1 minute, and a final extension at 68° C. for 8 minutes. A second round of PCR was performed with 1 ml of the first reaction mixture and the PCR anchor primer and the H30 and H5 primers (Table 1). The PCR products were cloned into the pCRII TOPO plasmid using the TOPO TA cloning kit from Clontech (Carlsbad, Calif., USA).

Construction of HCV-S1 cDNA Clones Encoding the Structural Proteins:

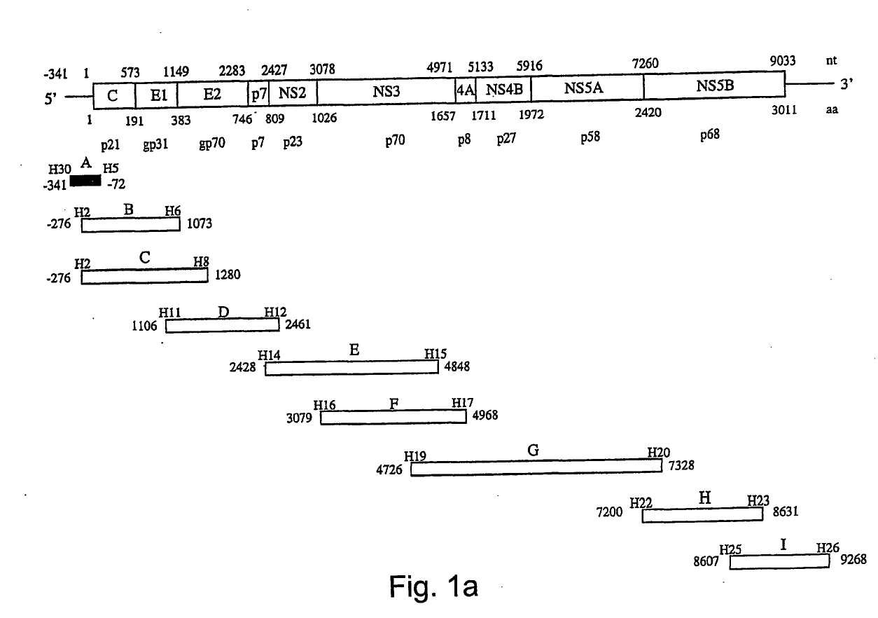

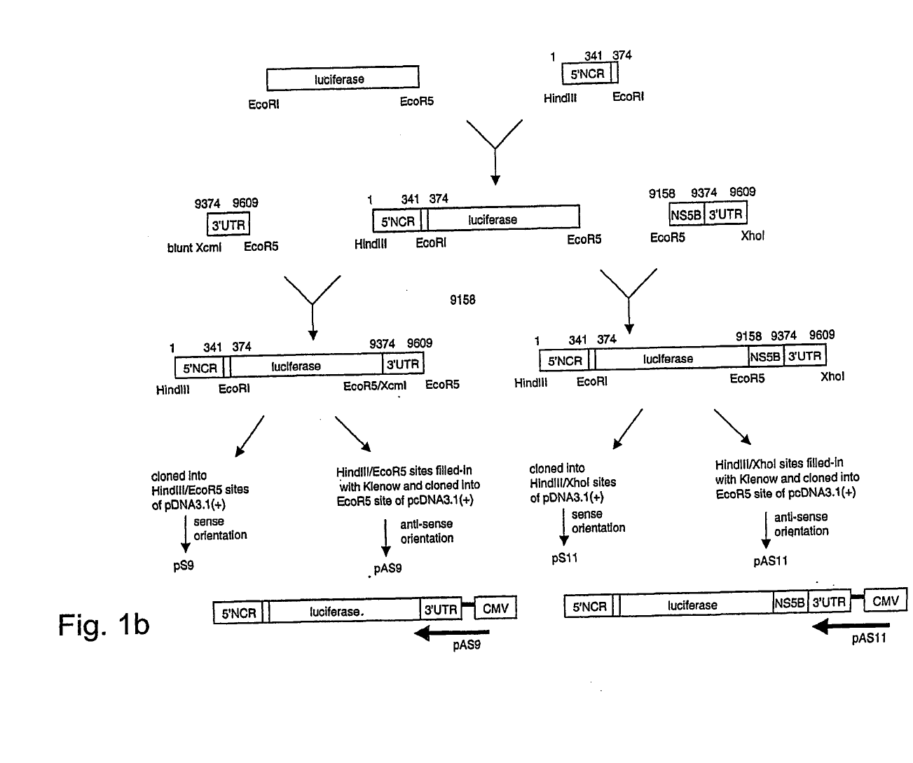

The region spanning the 5′ non-coding region (NCR) including the p7 region (nucleotides −276 to 2461 in FIG. 1A) was PCR amplified using clones C and D as templates and primers H2 and H12 (Table 1). The resulting 2.7 kb PCR product (nucleotides 65-2802 of SEQ ID NO:33) and a 600 bp PCR product comprising the NS2 cDNA (nucleotides 2769-3369 of SEQ ID NO:33) were used as templates for the H2 and H32 primers in a second round of PCR amplification (Table 1) to produce a 3.3 kb DNA fragment (nucleotides 65-3114 of SEQ ID NO:33). This PCR product and clone A were used as templates in a third round of PCR with primers H30 and H32. The resultant PCR product (nucleotides 1-3114 of SEQ ID NO: 33) was cloned into pXL TOPO TA vector from Clontech (Carlsbad, Calif., USA) to generate clone J (FIG. 1A). The truncated NS2 PCR product was amplified from clone E (FIG. 1A) using the primers H31 and H32. The PCR conditions were as follows: hot-start at 95° C. for 3 min, denaturation at 95° C. for 1 minute, annealing at 60-65° C. for 1 minute, and extension at 68° C. for 1 minute per 1 kb of amplified cDNA. At the end of 30 cycles, a final extension step was carried out at 68° C. for 8 minutes. Clone J was digested with EcoRI and re-cloned into pcDNA3.1(+) (Invitrogen) and pXJ41neo (Gift from C. Pallen, IMCB, 20) and correctly oriented clones were selected.

TABLE 1
Sequences of primers used for PCR amplification of overlapping
cDNA regions of the genome of HCV isolate HCV-S1.

| Primer | Sequence (5′-3′) | Position | Sense/Anti-sense | Reference |
|---|---|---|---|---|
| H1 | ACTGTCTTCACGCAGAAAGCGTCTAGC | −285 to −256 | sense | Bukh et al. |
| | CAT (SEQ ID NO: 1) | | | 1992 |
| H2 | CACGCAGAAAGCGTCTAGCCAT (SEQ | −276 to −247 | sense | Bukh et al. |
| | ID NO: 2) | | | 1992 |
| H3 | CGAGACCTCCCGGGGCACTCGCAAGCA | −14 to −43 | anti-sense | Bukh et al. |
| | CCC (SEQ ID NO: 3) | | | 1992 |
| H4 | TCCCGGGGCACTCGCAAGCACCCTATC | −21 to −50 | anti-sense | Bukh et al. |
| | AGG (SEQ ID NO: 4) | | | 1992 |
| H5 | CTATCAGGCAGTACCACAAGGCCTTTC | −43 to −72 | anti-sense | — |
| | GCG (SEQ ID NO: 5) | | | |
| H6 | CCCGCYAGGACYCCCCAGTGG (SEQ ID | 1073 to 1053 | anti-sense | Dirsel et al. |
| | NO: 6) | | | 1994* |
| H7 | GCCCAGTTCCCCACCATGGA (SEQ ID | 1106 to 1087 | anti-sense | Dirsel et al. |
| | NO: 7) | | | 1994* |
| H8 | AGGGCAGTCCTGTTGATGTGC (SEQ ID | 1280 to 1260 | anti-sense | Dirsel et al. |
| | NO: 8) | | | 1994* |
| H9 | AGGCTATCATTGCAGTTCAGGGC (SEQ | 1298 to 1276 | anti-sense | — |
| | ID NO: 9) | | | |
| H10 | CCACTGGGGRGTCCTRGCGGG (SEQ ID | 1053 to 1073 | sense | Dirsel et al. |
| | NO: 10) | | | 1994* |
| H11 | TCCATGGTGGGGAACTGGGC (SEQ ID | 1087 to 1106 | sense | Dirsel et al. |
| | NO: 11) | | | 1994* |
| H12 | CGCCTCCGCACGATGCAGCCAT (SEQ ID | 2461 to 2440 | anti-sense | — |
| | NO: 12) | | | |
| H13 | AGTACCAGACCTATGAAAACCGC (SEQ | 2483 to 2461 | anti-sense | — |
| | ID NO: 13) | | | |
| H14 | ATGGACCGGGAGATGGCTGCA (SEQ ID | 2428 to 2448 | sense | — |
| | NO: 14) | | | |
| H15 | AGGCTTTAGCCGTGTGAGACA (SEQ ID | 4848 to 4828 | anti-sense | — |
| | NO: 15) | | | |
| H16 | GCGCCYATCACGGCCTACTCC (SEQ ID | 3079 to 3099 | sense | — |
| | NO: 16) | | | |
| H17 | GACGACCTCCAGGTCAGCCGA (SEQ ID | 4968 to 4948 | anti-sense | — |
| | NO: 17) | | | |
| H18 | ACGCCCACTTCTTGTCTCAGA (SEQ ID | 4706 to 4726 | sense | — |
| | NO: 18) | | | |
| H19 | ACTAAGCAGGCAGGAGACAAC (SEQ ID | 4726 to 4746 | sense | — |
| | NO: 19) | | | |
| H20 | TTGATGGGTAATTTGCTCTCC (SEQ ID | 7328 to 7308 | anti-sense | — |
| | NO: 20) | | | |
| H21 | GTGGTGACGCAGCAAGGAGTT (SEQ ID | 7359 to 7339 | anti-sense | — |
| | NO: 21) | | | |
| H22 | CAGCGACGGGTCTTGGTCTAC (SEQ ID | 7200 to 7220 | sense | — |
| | NO: 22) | | | |
| H23 | TCACCGGTTGGGGAGCAGATAG (SEQ | 9033 to 9012 | anti-sense | — |
| | ID NO: 23) | | | |
| H24 | TCTACGGGGCCTACTACTCCATT (SEQ | 8597 to 8619 | sense | — |
| | ID NO: 24) | | | |
| H25 | CTACTACTCCATTGAGCCACTTGAC | 8607 to 8631 | sense | — |
| | (SEQ ID NO: 25) | | | |
| H26 | ACATGATCTGCAGAGAGGCCAGTATCA | 9269 to 9234 | anti-sense | Tanaka et al. |
| | GCACTCTC (SEQ ID NO: 26) | | | 1996 |
| H27 | GTCAAGTGGCTCAATGGAGTAGTAGGC | 8631 to 8605 | anti-sense | — |
| | (SEQ ID NO: 27) | | | |
| H28 | GCCAGCCCCCGATTGGGGGCGACACTC | −341 to −256 | sense | — |
| | CACCATAGATCACTCCCCTGTGAGGAA | | | |
| | CTACTGTCTTCACGCAGAAAGCGTCTA | | | |
| | GCCA (SEQ ID NO: 28) | | | |
| H29 | GACCACGCGTATCGATGTCGACTTTTTT | — | sense | 5′/3′ RACE kit |
| | TTTTTTTTTTV (SEQ ID NO: 29) | | | |
| H30 | GACCACGCGTATCGATGTCGAC (SEQ ID | — | sense | 5′/3′ RACE kit |
| | NO: 30) | | | |
| H31 | ATGGACCAGGAGTTGGCTGCATCGTGC | 2769 to −2796 | sense | — |
| | (SEQ ID NO: 31) | | | |
| H32 | CTAACGCGCACGCACGAATGAGGCCTT | 3115 to 3008 | anti-sense | — |
| | (SEQ ID NO: 32) | | | |

Construction of HCV-S1 cDNA Clones Encoding the NS Proteins:

The region spanning NS3 to NS5A (nucleotides 3420-7669 of SEQ ID NO:33) was obtained by double-cloning a 1.844 kb BamHI/BmrI fragment (nucleotides 3420-5263 of SEQ ID NO:33) from clone F (FIG. 1A) and a 2.4 kb BmrI/EcoRV fragment (nucleotides 5263-7669 of SEQ ID NO:33) from clone G (FIG. 1A) into pKSII (±) digested with BamHI and EcoRV. The resulting clone was digested with XbaI and BsrGI and ligated to a 0.9 kb XbaI/BsrGI fragment (nucleotides 2769-3640 of SEQ ID NO:33) containing the NS2 ORF from clone E, to thereby produce clone K (FIG. 1A). To generate the region spanning nucleotides 7200 to 9268 of the HCV genome, clones H and I (FIG. 1A) were used as templates in a PCR reaction with primers H22 and H26 (Table 1). The resultant PCR product (nucleotides 7641-9609 of SEQ ID NO:33) was cloned into pCRIITOPO to generate clone L (FIG. 1A). Clones K and L were each introduced into electro-competent GM109 bacteria cells and DNA plasmids preparations of these clones were digested with BclI and EcoRV and co-ligated to generate clone M (FIG. 1A). Clone M was digested with NotI and XhoI and re-cloned into pcDNA3.1(+) and pXJ41neo to generate pcDNA3(NSP) and pXJ41(NSP) respectively.

Construction of Full-Length cDNA Clones of HCV-S1:

Clones J and M were digested with CspI and XbaI and the resulting 3.3 kb fragment from clone J (nucleotides 1-3369 of SEQ ID NO:33) including the anchor-5′NCR to NS2 sequence was ligated into clone M to generate a full length genome of HCV-S1 in pKSII(±) (designated pKSII(S1)). To generate the full length clone in pcDNA3.1(+), the EcoRV/BsrGI fragment from pKSII(S1) was ligated to the pcDNA3(NSP) digested with the same enzymes to generate pcDNA3(S1). The same fragment was cloned into the blunt-NotI/Bsrgl site in pXJ41(NSP) to generate pXJ41(S1).

Renilla Luciferase Expression Construct:

The renilla luciferase cDNA (GeneBank Accession number M63501, nucleotide coordinates 10-945) including the upstream intron sequence from human growth hormone (GeneBank Accession number M13438, nucleotide coordinates 569-827) was PCR amplified from pBIND (Promega) and subcloned into the HindIII site of pcDNA3.1(+). Clones containing the insert in the right orientation were isolated and verified by sequence analysis.

Chimeric HCV-Luciferase Constructs:

The firefly luciferase gene (GeneBank Accession number M15077, nucleotide coordinates 253-2387) was PCR amplified from the plasmid pGL3-Basic (Promega, Madison, Wis.). The PCR product was digested with EcoRI and EcoRV and re-cloned into pcDNA3.1(+) (Clontech) to generate the construct pLUCEE(15). The HCV sequence from nt 1-374 comprising the full length 5′NCR and the first 33 nt of its core sequence (nucleotides 1-374 of SEQ ID NO:33) was PCR amplified from HCV-S1. The PCR product was digested with HindIII and EcoRI and cloned into pLUCEE 15 to generate the construct pLUCEE15NC(B2). In order to clone the entire 3′UTR of HCV-S1 downstream of pLUCEE15NC(B2), the plasmid pHCV700(A8) (clone I, FIG. 1A) was digested with XcmI and EcoRV and blunted with Klenow. The resultant insert was cloned into the EcoRV site of pLUCEE15NC(B2) and clones with the 3′UTR cloned in the right orientation were isolated. One of these clones pLUCNC3UTR(B9) was excised with HindIII and XhoI, blunted with Klenow and cloned into the EcoRV site of pcDNA3.1(+). Clones with inserts in the anti-sense orientation were isolated and designated pAS9 (FIG. 1A). Next, chimeric HCV-luciferase constructs which contained HCV NS5B and 3′UTR sequences were generated. A region covering the C-terminal end of the NS5B sequence and the complete 3′UTR of HCV-S1 was PCR amplified from pHCV700(A8) (clone I, FIG. A). The PCR product (nucleotides 9159-9609 of SEQ ID NO:33) was digested with EcoRV and XhoI and cloned into pLUCEE15NC(B2) to generate pLUCNC5BUTR(11). The insert from this construct was excised with HindIII and XhoI, blunted with Klenow and cloned into the EcoRV site of pcDNA3.1(+). Clones with inserts in the anti-sense orientation were isolated and named pAS 11 (FIG. 1A). All constructs were verified via enzymatic restriction digestions and sequence analyses. FIG. 1B illustrates the above described steps utilized in generating the chimeric anti-sense expression constructs pAS9 and pAS21 and their sense oriented counterparts.

Sequence Analysis:

DNA sequencing of all constructs was carried out using the Taq DyeDeoxy terminator cycle sequencing kit and an automated DNA sequencer 373 from PE Applied Biosystems (Foster City, Calif., USA).

Cells and Cell Culture:

The human embryonic kidney cell line, 293, its derivative, 293T, which bears the large T antigen from SV40, and the human hepatoma cell line HuH-7 were all purchased from American Type Cell Collection (ATCC). The cells were cultured in Dulbecco's Minimal Essential Media (DMEM) containing 2 mM L-glutamine, and 10% fetal bovine serum and maintained at 37° C. in 5% CO₂.

Cell Transfections:

Transfections were performed using the Effectene™ transfection reagent from QIAGEN (Valencia, Calif., USA). Approximately 2×10⁵ cells were plated into 6-well tissue culture plates 14-18 hours prior to transfection. A total of 1 μg of plasmid DNA in 150 μl EC buffer was mixed with 8 ill of enhancer and vortexed for 10 seconds. The mixture was allowed to stand at RT for 2-5 minutes, 25 μl of Effectene™ transfection reagent was added, the mixture vortexed again and incubated at RT for another 5-10 minutes. Cells were washed with PBS, added into DNA-Effectene™ mixture diluted in 2 ml of complete growth medium and incubated at 37° C. and 5% CO₂ for 6-8 hours. Following incubation, the medium was removed and the cells were washed with once with PBS. Approximately 2.5 ml of fresh complete medium was added to the cells and the cells were incubated for an aditional 48-120 hours, following which cells were harvested for RNA isolation or western analysis, or treated with 1000 mg/ml G418 for selection of stable clones.

Luciferase Assays:

Luciferase activity was measured using the a luciferase assay kit (Promega, Madison, Wis.). Following a 72-120 hour incubation period, cells were washed twice with PBS and lysed with 100 μl reporter lysis buffer (Promega). The lysate was allowed to stand at room temperature for 10-15 minutes. Following which, the lysate was centrifuged for 1 min in a microfuge and a 10 μl aliquot was mixed with 100 μl of reporter buffer (Promega); luciferase activity was measured in a Turner luminometer (Turner Designs, Sunnydale, Calif.) over an integration period of 15 seconds. In cells co-transfected with pCMV-Ren, cell pellets were re-suspended in 100 ml of passive lysis buffer and measured using the dual-luciferase system from Promega. Values obtained were normalized with the levels of Renilla luciferase activity in the cell lysates and the total protein concentration.

In-Vitro Translation:

Translation was effected via the TNT quick coupled transcription/translation system from Promega. Briefly, 0.5-1 μg of plasmid DNA was mixed with 40 μl of TNT quick master mix and 2 μl of ³⁵S methionine (10 mCi/ml) (NEN). The reaction mixture was incubated at 30° C. for 1-3 hours. Following a predetermined time period, an aliquot was removed and SDS-Page analysis was performed. Where indicated, between 0.3-2.5 μl of canine pancreatic microsomal membranes (Promega) were added to the reaction mixture.

Western Blot Analysis:

Cell lysates were resolved on a 10 or 12% sodium dodecyl sulphate (SDS)-polyacrylamide gel, transferred to a nitrocellulose membrane, blocked with 5% nonfat skim milk in PBS, and incubated with a primary antibody followed by incubation with anti-mouse or anti-human secondary antibody conjugated to horseradish peroxidase (Sigma). Detection was effected using the ECL enhanced chemiluminescence kit (Pierce). The E2 directed antibody (H52), was a kind gift from J. Dubuisson (Institut de Biologie de Lille & Institut Pasteur de Lille, Lille Cedex, France). The NS3 and NS5A directed monoclonal antibodies were purchased from Devaron, Inc. (NJ, USA) and Biodesign International (ME, USA) respectively.

### Experimental Results

Generation of HCV Overlapping cDNA Clones:

Sera derived from a single chronic HCV carrier were subjected to RT-PCR, and nine overlapping cDNAs clones covering the entire HCV genome were (FIG. 1A). The overlapping regions in these clones had almost identical sequences (data not shown). To obtain the complete 5′ NCR sequence of this isolate, 5′ rapid amplification of cDNA ends was effected using the 5′/3′ RACE kit from Boehringer Mannheim. Following two rounds of nested PCR, a cDNA fragment comprising the 5′ NCR region spanning nucleotides −341 to −72 that was missing from clones B and C was obtained. The overlapping cDNA clones of isolate HCV-S1 span 9609 nucleotides encoding a complete polyprotein 3010 amino acids long (SEQ ID NO:34), and a 341-nt 5′ NCR, and a 235-nt 3′ NCR (FIG. 1A). To determine the genotype of isolate HCV-S1, the sequence of a region of 226 nt within the 5′ NCR (from −276 to −21, FIG. 1A) (2) as well as 233 nt within NS3 (from 4699 to 4932, FIG. 1A) and 400 nt within NS5B (from 7904 to 8304, FIG. 1A) (5) were analyzed. Following comparison to available HCV sequences, it was determined that HCV-S1 belongs to the type 1 genotype, with a 1b subtype. Sequence comparisons of the other two regions were consistent with this finding.

Characterization of Full Length HCV Genome:

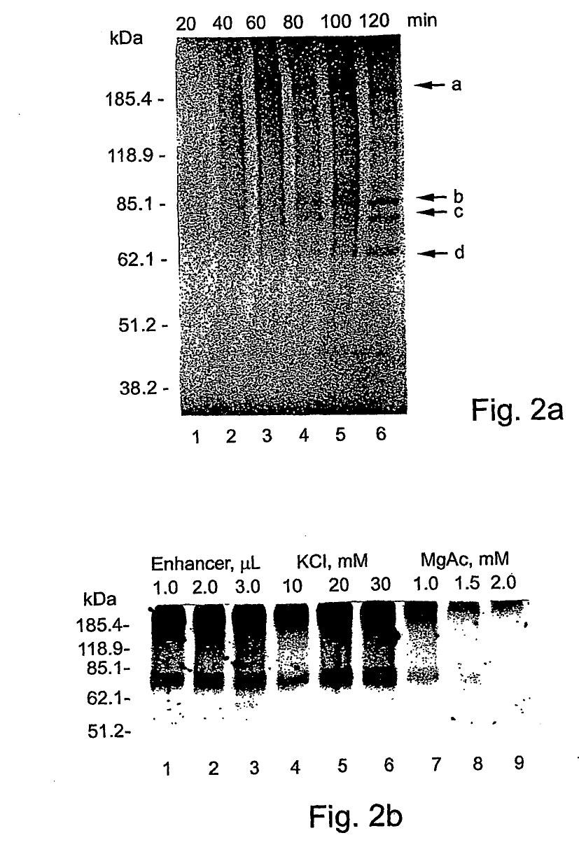

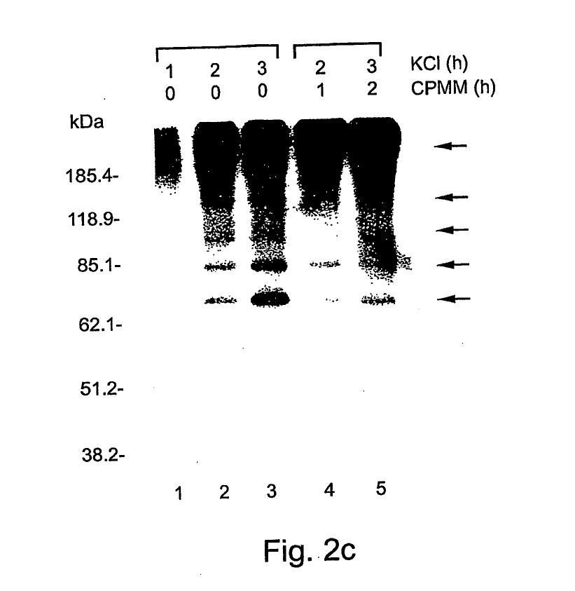

The full length HCV genome was generated as described hereinabove to produce pcDNA3(S1) and pXJ41(S1) respectively. To characterize this clone, in vitro coupled transcription and translation was first carried out with pcDNA3(SP) and pcDNA3(NSP) using a kit from Promega. A single polyprotein larger than 185 kD was observed following one hour of incubation with pcDNA3(NSP) (FIG. 2A, lane 2). Prolonged incubation periods gave rise to smaller protein products (FIG. 2A, lanes 4-6). Following two hours of incubation, distinct bands corresponding to proteins of approximately 80, 75 and 62 kD in size were also detected (FIG. 2A, lane 6). It is believed that these products are the result of the enzymatic activity of the protease moiety of NS3 and as such these bands possibly correspond to NS3-4A (77 kD), NS5B (68 kD) and NS5A (58 kD).

The construct pcDNA3(SP) contains the entire HCV sequence of the core, E1 and E2 proteins, and the first 115 amino acids of NS2 and as such when translated should give rise to a polyprotein of about 82 kD. In vitro translation experiments with this construct with addition of either an enhancer or KCl produced a single band corresponding to about 82 kD (FIG. 2B, lanes 1-6), whilst addition of magnesium acetate failed to produce any band (FIG. 2B, lanes 7-9).

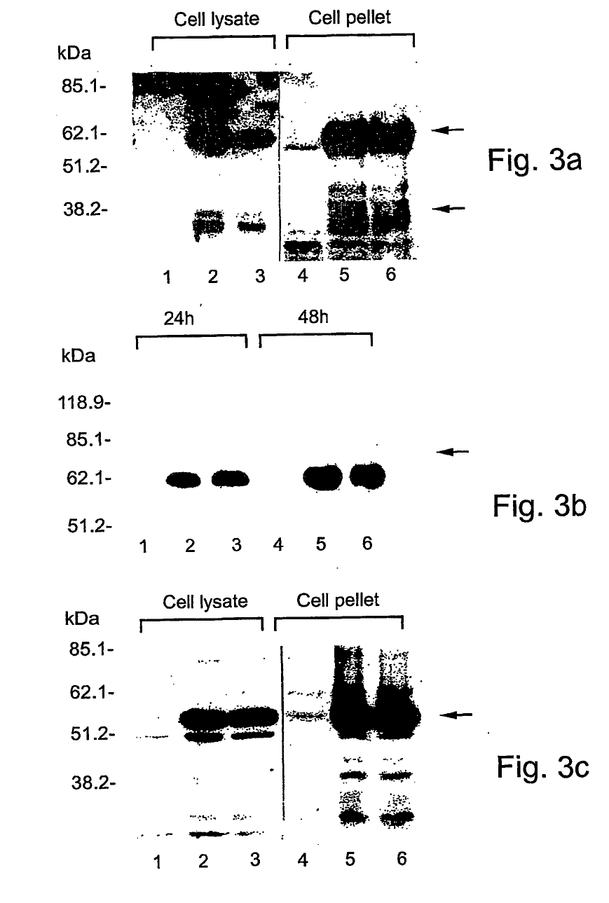

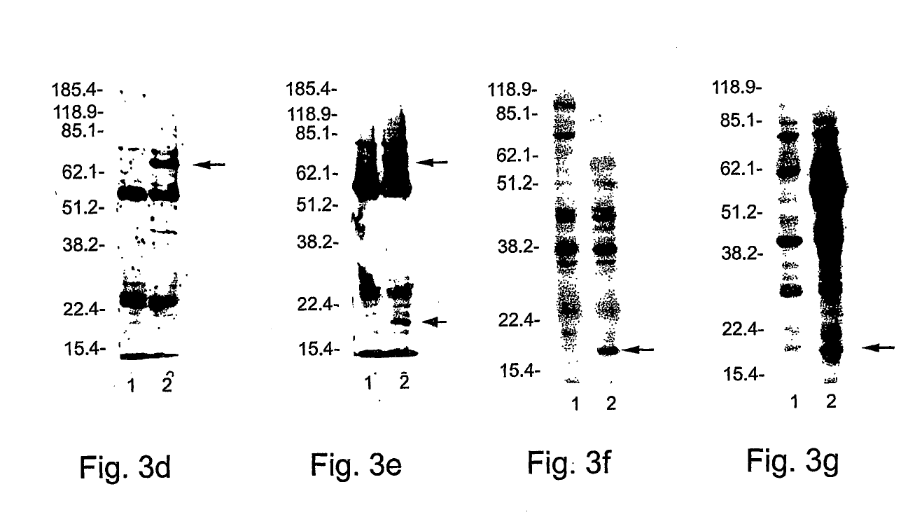

The above described was repeated with the pcDNA3(S1) construct. Following a one hour incubation, a broad band larger than 185 kD was observed (FIG. 2C, lane 1). Following two hours of incubation, several smaller bands were observed of sizes ranging from 65 to 140 kD. In addition, two fainter bands of 60 kD and 50 kD were also detected (FIG. 2C, lane 2). The intensity of the bands increased slightly when incubation was allowed to proceed for three hours (FIG. 2C, lane 3). This suggests that the HCV polyprotein was proteolytically cleaved in vitro, mostly likely by the NS3 protease. Interestingly addition of canine pancreatic microsomal membranes (CPMM) led to disappearance of the two upper bands of about 140 and 100 kD and reduction in intensity of the lower two bands (FIG. 2C, lanes 4 and 5). It is likely that these bands represent subfragments of the HCV polypeptide and were post-translationally processed by the microsomal vesicles. pXJ41(S1) was transiently transfected into 293T cells, and the expression of HCV proteins was examined. Structural (core and E2) and non-structural (NS3, NS5A, NS5B) proteins (FIGS. 3A-G) were detected using available monoclonal or polyclonal antibodies.

These results indicate that the full length HCV genome cloned while reducing the present invention to practice, is able to direct the expression of the full length polyprotein and is capable of being processed.

Results of Transfection of Anti-Sense Chimeric HCV-Luciferase Construct pASB9:

The 293T and HuH7 cell lines were separately transfected with two different clones of pASB9 (pASB9.1 and pASB9.2), which contain an anti-sense chimera of the firefly luciferase gene downstream of a HCV 5′ NCR-core sequence and upstream of the HCV 3′ UTR sequence. Transfection was carried out with pASB9 and an equal amount of pXJ41(NSP), pXJ41(S1) or a combination of pXJ41(NS3) and pXJ41(NS5B). Co-transfection with the vector, pXJ41neo was used as a control to measure background luciferase activity. The cells were harvested and assayed for luciferase activity 5 days post-transfection. There was no observed increase in luciferase activity in co-transfection experiments with any of the HCV expression constructs compared to co-transfection with the vector (data not shown). Experiments carried out with a total of 1 or 2 ? g of DNA produced similar results.

Results of Transfection of Anti-Sense Chimeric HCV-Luciferase Construct pAS11:

Similar experiments were carried with the anti-sense construct pAS11 (pAS 11-12 and pAS 11-15) which contains the anti-sense chimera of the firefly luciferase gene downstream of a HCV 5′ NCR-core sequence and upstream of the HCV NS5B-3′ UTR sequence. In 293T cells, co-transfection with the full length HCV expression plasmid, pXJ4l(S1) and pAS11-12 produced a 10-fold increase over background luciferase activities five days post transfection, while a 14.7-fold increase was observed with pAS11-15 co-transfected with pXJ41(S1) (Table 2). In similarly transfected HuH7 cells, luciferase activities were 2.7-fold and 5.8-fold above background values three days post transfection (Table 3). At five days post transfection, the luciferase activities in HuH7 cells slightly increased to 3.7-fold and 6.2-fold respectively (Table 4). However, co-transfection of pAS11-12 or -15 with the NS proteins expression vector (pXJ41(NSP)) or the vector including NS3 and NS5B, resulted in no detectable increase in luciferase activity as compared to transfection with vector alone (Tables 2-4).

TABLE 2

| | Constructs | R1 | R2 | Av | |
|---|---|---|---|---|---|
| 1 | pAS11(12) + pXJ41neo | 1.20 | 1.06 | 1.13 | |
| 2 | pAS11(12) + pXJ41(NS3) + | 1.22 | 1.46 | 1.34 | |
| | pXJ41(NS5B) | | | | |
| 3 | pAS11(12) + pXJ41(NSP) | 1.40 | 1.30 | 1.35 | |
| 4 | pAS11(12) + pXJ41(S1) | 12.34 | 11.11 | 11.73 | 10.36X |
| 5 | pAS11(15) + vector | 1.39 | 1.46 | 1.42 | |
| 6 | pAS11(15) + pXJ41(NS3) + | 1.92 | 1.53 | 1.73 | |
| | pXJ41(NS5B) | | | | |
| 7 | pAS11(15) + pXJ41(NSP) | 1.31 | 1.33 | 1.32 | |
| 8 | pAS11(15) + pXJ41(S1) | 21.79 | 20.09 | 20.94 | 14.7X |
| 9 | p11(3) + pXJ41neo | 5106.2 | 5082.7 | 5094.5 | |
| 10 | p11(6) + pXJ41neo | 5611.5 | 5440.0 | 5525.8 | |
| 11 | pXJ41neo | 0 | −0.2 | 0 | |

p11(3) - sense, clone 11
p11(6) - sense, clone p11
R - luciferase reading
Av - average luciferase reading

TABLE 3

| | Constructs | R1 | R2 | Av | |
|---|---|---|---|---|---|
| 1 | pAS11(12) + pXJ41neo | 5.52 | 5.27 | 5.40 | |
| 2 | pAS11(12) + pXJ41(NS3) + pXJ41(NS5B) | 3.10 | 2.98 | 3.04 | |
| 3 | pAS11(12) + pXJ41(NSP) | 4.11 | 4.12 | 4.11 | |
| 4 | pAS11(12) + pXJ41(S1) | 15.21 | 14.26 | 14.74 | 2.73X |
| 5 | pAS11(15) + vector | 3.25 | 3.70 | 3.47 | |
| 6 | pAS11(15) + pXJ41(NS3) + pXJ41(NS5B) | 3.15 | 3.23 | 3.19 | |
| 7 | pAS11(15) + pXJ41(NSP) | 3.24 | 3.14 | 3.19 | |
| 8 | pAS11(15) + pXJ41(S1) | 19.87 | 20.07 | 19.97 | 5.76X |
| 9 | p11(3) + pXJ41neo | 10348.8 | 9848.0 | 10098.4 | |
| 10 | p11(6) + pXJ41neo | 13418.2 | 12821.6 | 13119.9 | |
| 11 | pXJ41neo | 0 | 0 | 0 | |

p11(3) - sense, clone 11
p11(6) - sense, clone p11
R - luciferase reading
Av - average luciferase reading

TABLE 4

| | Constructs | R1 | R2 | Av | |
|---|---|---|---|---|---|
| 1 | pAS11(12) + pXJ41neo | 2.03 | 2.09 | 2.06 | |
| 2 | pAS11(12) + pXJ41(NS3) + | 0.82 | 0.66 | 0.74 | |
| | pXJ41(NS5B) | | | | |
| 3 | pAS11(12) + pXJ41(NSP) | 1.36 | 1.09 | 1.22 | |
| 4 | pAS11(12) + pXJ41(S1) | 8.26 | 6.98 | 7.62 | 3.7X |
| 5 | pAS11(15) + vectar | 2.19 | 2.02 | 2.10 | |
| 6 | pAS11(15) + pXJ41(NS3) + | 0.92 | 0.79 | 0.86 | |
| | pXJ41(NS5B) | | | | |
| 7 | pAS11(15) + PXJ41(NSP) | 0.63 | 0.61 | 0.62 | |
| 8 | pAS11(15) + pXJ41(S1) | 13.89 | 12.28 | 13.09 | 6.23X |
| 9 | p11(3) + pXJ41neo | 3565.8 | 3450.3 | 3508.1 | |
| 10 | p11(6) + pXJ41neo | 4197.2 | 3861.9 | 4030 | |
| 11 | pXJ41neo | 0 | 0 | 0 | |

p11(3) - sense, clone 11
p11(6) - sense, clone p11
R - luciferase reading
Av - average luciferase reading

Results of Co-Transfection with pAS11 and pCMV-Ren:

Similar experiments were conducted using a renilla expression construct pCMV-Ren, in order to account for any variation in luciferase activity due to different transfection efficiencies. 293T and HuH7 cells were transfected with a total of 1 μg of DNA and cells were harvested and analyzed 3 days post-transfection. All values obtained were normalized against total protein concentration and renilla luciferase activity. In 293T cells, co-transfection of pASI 1-15 with pXJ41(S1) resulted in a 18.5-fold increase over background luciferase activity (Table 5). In HuH7 cells, the luciferase activity of pAS11-15 was 3.9-fold higher when co-transfected with pXJ4 1 (S1) (Table 6).

TABLE 5

| | | 5 × dilution | 5 × dilution | | 5 × dilution | Neat | Neat |
|---|---|---|---|---|---|---|---|
| | Construts | Av FF LUC | Av REN | N. Ren (X) | N. Av FFL | Av FFL | Total FFL |
| 1 | pAS11(12) + pXJ41neo | 0.39 | 1491 | 1.00 | 0.39 | 8.56 | 8.56 |
| 2 | pAS11(12) + pXJ41(S1) | 0.72 | 478 | 3.12 | 2.25 | 145.80 | 158.146 |
| 3 | AS11(3) + pXJ41neo | 37.13 | 1626 | 0.92 | 34.16 | 9538.00 | 11445.6 |

| | Constructs | Prot. Conc | PN (X) | Total FFL | Final FFL | |
|---|---|---|---|---|---|---|
| 1 | pAS11(12) + pXJ41neo | 0.303 | 1 | 8.56 | 8.56 | |
| 2 | pAS11(12) + pXJ41(S1) | 0.284 | 1.07 | 145.80 | 158.15 | 18.48X |
| 3 | AS11(3) + pXJ41neo | 0.253 | 1.2 | 9538.00 | 11445.60 | |

Av FF LUG = average of 2 firefly luciferase readings from 20 ml of 5 × diluted cell lysate
Av REN = average of 2 renilla luciferase readings from 20 ml of 5 × diluted cell lysate
N. Ren (X) = normalisation index of renilla luciferase readings from 20 ml of 5 × diluted cell lysate
N. Av FFL = average of 2 firefly luciferase readings from 20 ml of 5 × diluted cell lysate after normalisation against renilla luciferase index
Av FFL = normalised average of 2 firefly luciferase readings from 20 ml of cell lysate (neat)
Final Total FFL = normalised average of 2 firefly luciferase readings of total cell lysate
PN (X) = protein normalisatlon index
Final FFL = final average of 2 firefly luciferase readings of total cell lysate

TABLE 6

| | | 5 × dilution | 5 × dilution | | 5 × dilution | Neat | Neat |
|---|---|---|---|---|---|---|---|
| | Constructs | Av FF LUC | Av REN | N. Ren (X) | N. Av FFL | Av FFL | Total FFL |
| 1 | pAS11(12) + pXJ41neo | 0.39 | 1491 | 1.00 | 0.39 | 1.97 | 7.86 |
| 2 | pAS11(12) + pXJ41(S1) | 0.72 | 478 | 3.12 | 2.25 | 11.23 | 44.92 |
| 3 | AS11(3) + pXJ41neo | 37.13 | 1626 | 0.92 | 34.16 | 17.08 | 683.20 |

| | Constructs | Prot. Conc | PN (X) | Total FFL | Final FFL | |
|---|---|---|---|---|---|---|
| 1 | pAS11(12) + pXJ41neo | 0.063 | 1 | 7.86 | 7.86 | |
| 2 | pAS11(12) + pXJ41(S1) | 0.093 | 0.68 | 44.92 | 30.55 | 3.88X |
| 3 | pAS11(3) + pXJ41neo | 0.106 | 0.59 | 683.20 | 403.09 | |

Av FF LUC = average of 2 firefly luciferase readings from 20 ml of 5 × diluted cell lysate
Av REN = average of 2 renilla luciferase readings from 20 ml of 5 × diluted cell lysate
N. Ren (X) = normalisation index of renilla luciferase readings from 20 ml of 5 × diluted cell lysate
N. Av FFL = average of 2 firefly luciferase readings from 20 ml of 5 × diluted cell lysate after normalisation against renilla luciferase index
Av FFL = normalised average of 2 firefly luciferase readings from 20 ml of cell lysate (neat)
Final Total FFL = normalised average of 2 firefly luciferase readings of total cell lysate
PN (X) = protein normalisation index
Final FFL = final average of 2 firefly luciferase readings of total cell lysate

Cells co-transfected with pASB9 with different HCV expression constructs failed to produce changes in luciferase activity (data not shown). However, pAS 11 consistently produced increased luciferase activities when co-transfected with pXJ41(S1), which expresses the full length HCV genome. In 293T cells, the levels were between 10.4-14.7 folds above background levels, and in HuH7 cells they were between 2.7-6.2 folds (Tables 2-4). Even after normalizing with co-transfection with a plasmid that expresses renilla luciferase, a significant increase in luciferase activities was observed. In 293T cells, the increase was 17-fold above background, while in HuH7 cells, it was 3.9-fold (Tables 5 and 6). These results indicate that the additional C-terminal NS5B coding sequence present only in pAS11 is important and necessary for the NS5B polymerase (and perhaps other factors) to bind efficiently and initiate reverse strand synthesis.

Several reports have shown that in vitro provided NS5B is capable of binding and initiating the synthesis of sequences containing the 3′ UTR alone (17, 18). Yet, the experiments conducted while reducing the present invention to practice clearly indicate that the 3′ UTR alone is insufficient in promoting polymerase activity in vivo. As such, this is the first demonstration that the NS5B region works together with the 3′ UTR to facilitate negative strand synthesis in vivo.

Interestingly co-transfection with an expression vector for the non-structural proteins, pXJ41(NSP) or with expression vectors for NS3 and NS5B did not result in any increase in luciferase activity when compared to co-transfection with the vector alone. This suggests that the synthesis of the sense strand HCV-luciferase chimeric RNA by the HCV NS5B polymerase is dependent on multiple viral proteins, including both non-structural and viral protein(s). It also indicates that a full length replication-competent HCV genome is required for this assay to be functional.

This is the first demonstration that negative strand synthesis depends on expression of essentially all the viral proteins in intact cells. Based on these findings, the present invention provides a cell-based HCV replication-dependent system that is a measure of the activity of the full-length HCV genome. This system is simple, and robust and highly reproducible and in addition, enables to measure viral activity as early as three days post-transfection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences disclosed therein and/or identified by a GeneBank accession number mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

References

1. Houghton M, Weiner A, Han J, Kuo. G and Choo Q L 1991 Hepatology 14: 381-388.

2. Alter H J, Purcell R H, Shih J W, Melpolder J C, Houghton M, Choo Q L, Kuo G (1989) Detection of antibody to hepatitis C virus in prospectively followed transfusion receipients with acute and chronic non-A, non-B hepatitis. N Engl. J Med. 321: 1491-1500.

3. Choo Q L, Richman K H, Han J H, Berger K, Lee C, Dong C, Gallegos C, Coit D, Medina S R, Barr P J (1991) Genetic organization and diversity of the hepatitis virus. PNAS 88: 2451-2455.

4. (4) Harada S, Watanabe Y, Takeuchi K, Suzuki T, Katayama T, Takebe Y, Saito et al. 1991 J Virol. 65: 3015-3021.

5. (5) Hijikata M, Kato N, Ootsuyama Y, Nakagawa M, Shimotohno K 1991. Gene mapping of the putative structural region of the hepatitis C virus genome. PNAS 88: 5547-5555.

6. Matsura Y, Harada S, Suzuki R, Watanabe Y, Inoue Y, Saito , Miyamura T 1992. Expression of processed envelope protein hepatitis C virus in mammalian and insect cells. J. Virol. 66: 1425-1431.

7. Tomei L, Failla C, Santolini E, De-Francesco R, La-Monica N 1993 NS3 is a serine protease required for processing of hepatitis C virus polyprotein. J. Virol. 67: 4017-4026.

8. Selby Mj, Choo Q L, Berger K. Kuo G, Glazer E., Eckart M, Lee C et al. 1993 Expression, identification and subcellular localisation of the proteins encoded by the hepatitis C viral genome. J. Gen Virol. 74: 1103-1113.

9. Shimizu Y K, Purcell R H, Yoshikura H. 1993. Correlation between the infectivity of hepatitis C virus in vivo and its infectivity in vitro. PNAS 90: 6037-6041.

10. Bertolini L, lacovacci S, Ponzetto A, Gorini G, Bataglia M, Carloni G. 1993 The humna bone marrow-derived B-cell line susceptible to hepatitis C virus infection. Res. Virol. 144: 281-285.

11. lacovacci S, Sargiacomo M, Parolini I, Ponzetto A, Peschle C, Carloni G. 1993 Replication and multiplication of hepatitis C virus genome in human fetal liver cells. Res Virol. 144: 275-279.

12. Ito T, Mukaigawa J, Zuo J, Hirabayashi Y, Mitamura K, Yasui K. 1996 Cultivation of hepatitis C virus in primary hepatocyte culture from patients with chronic hepatitis C results in release of high titer infectious virus. J. Gen Virol. 77: 1043-1054.

13. Lanford R E, Sureau C, Jacob J R, White R, Fuerst T R. 1994. Demonstration of in vitro infection of hepatocytes with hepatitis C virus using starnd-specific RT/PCR. Virology 202: 606-614.

14. Lohmann, V, Komer F, Koch J O, Herian U, Theilmann L, Bartenschlager R. 1999 Replication of subgenomic Hepatitis C virus RNAs in a hepatoma cell line. Science 285: 110-113.

15. Behrens S E, Tomei L, De Francesco R. 1996. Identification and properties of the RNA-dependent RNA polymerase of hepatitis C virus. EMBO J 2;15(1):12-22.

16. Sun X L, Johnson R B, Hockman M A, Wang Q M. 2000. De novo RNA synthesis catalyzed by HCV RNA-dependent RNA polymerase. Biochem Biophys Res Commun. 268(3):798-803.

17. Zhong W, Uss A S, Ferrari E, Lau J Y, and Z. Hong. 2000. De novo initiation of RNA synthesis by hepatitis C virus nonstructural protein 5B polymerase. J Virol 74(4):2017-22.

18. Oh J W, Sheu G T, and M M. Lai. 2000. Template requirement and initiation site selection by hepatitis C virus polymerase on a minimal viral RNA template. J Biol Chem. 2000 Apr. 3.

19. Zheng X M, Wang Y, Pallen C J. 1992 Cell transformation and activation of pp60c-src by overexpression of a protein tyrosine phosphatase. Nature. 359: 336-9.

What is claimed is:

1. A nucleic acid construct comprising:
(a) an expression cassette including:
(i) a first polynucleotide region including a 5′ NCR sequence of an RNA virus and at least an N-terminal portion of a coding sequence of said RNA virus;
(ii) a second polynucleotide region including a 3′ UTR sequence of said RNA virus and at least a C-terminal portion of a coding sequence of said virus; and
(iii) a third polynucleotide region encoding a reporter molecule, said third polynucleotide region being flanked by said first and said second polynucleotide regions; and
(b) a promoter sequence being operatively linked to said expression cassette in a manner so as to enable a transcription of a minus strand RNA molecule from said expression cassette.

2. The nucleic acid construct of claim 1, wherein at least a portion of said first polynucleotide region is at least 50% identical to a sequence encompassed by nucleotides 1-374 of SEQ ID NO:33.

3. The nucleic acid construct of claim 1, wherein at least a portion of said second polynucleotide region is at least 50% identical to a sequence encompassed by nucleotides 9158-9609 of SEQ ID NO:33.

4. The nucleic acid construct of claim 1, wherein said first polynucleotide region further includes a 5′ UTR sequence of said RNA virus.

5. The nucleic acid construct of claim 1, wherein said C-terminal portion of said coding sequence of said virus includes coding sequences of a polymerase of said virus.

6. The nucleic acid construct of claim 1, wherein said first polynucleotide region includes an IRES sequence.

7. The nucleic acid construct of claim 1, wherein said RNA virus is selected from the group consisting of a positive strand RNA virus and a negative strand RNA virus.

8. The nucleic acid construct of claim 1, wherein said RNA virus is selected from the group consisting of a virus of the picomavirus family, a virus of the togavirus family, a virus of the orthomyxovirus family, a virus of the paramyxovirus family, a virus of the coronavirus family, a virus of the calicivirus family, a virus of the arenavirus family, a virus of the rhabdovirus family and a virus of the bunyavirus family.

9. The nucleic acid construct of claim 1, wherein said RNA virus is Hepatitis C.

10. The nucleic acid construct of claim 1, wherein said first and said second polynucleotide regions are selected such that said minus strand RNA molecule transcribable from said expression cassette is replicatable by an RNA dependent RNA polymerase of said virus into a plus strand RNA molecule.

11. The nucleic acid construct of claim 1, wherein said promoter is functional in a eukaryotic cell.

12. The nucleic acid construct of claim 11, wherein said eukaryotic cell is selected from the group consisting of an insect cell, a yeast cell and a mammalian cell.

13. The nucleic acid construct of claim 1, wherein said reporter molecule is a polypeptide selected from the group consisting of an enzyme, a fluorophore, a substrate and a ligand.

14. A genetically transformed cell comprising a nucleic acid construct including:
(a) an expression cassette including:
(i) a first polynucleotide region including a 5′ NCR sequence of an RNA virus and at least an N-terminal portion of a coding sequence of said RNA virus;
(ii) a second polynucleotide region including a 3′ UTR sequence of said RNA virus and at least a C-terminal portion of a coding sequence of said virus; and
(iii) a third polynucleotide region encoding a reporter molecule, said third polynucleotide region being flanked by said first and said second polynucleotide regions; and
(b) a promoter sequence being operatively linked to said expression cassette in a manner so as to enable a transcription of a minus strand RNA molecule from said expression cassette.

15. The genetically transformed cell of claim 14, further comprising an additional nucleic acid construct for expressing at least an RNA dependent RNA polymerase of a virus, said first and said second polynucleotide regions being selected such that said RNA dependent RNA polymerase is capable of replicating said minus strand RNA molecule into plus strand RNA.

16. The genetically transformed cell of claim 14, wherein at least a portion of said first polynucleotide region is at least 50% identical to a sequence encompassed by nucleotides 1-374 of SEQ ID NO:33.

17. The genetically transformed cell of claim 14, wherein at least a portion of said second polynucleotide region is at least 50% identical to a sequence encompassed by nucleotides 9158-9609 of SEQ ID NO:33.

18. A method of detecting a presence of an RNA virus in a cell, the method comprising the steps of:
(a) incubating a nucleic acid construct with an extract of the cell under conditions suitable for transcription and translation of said nucleic acid construct, said nucleic acid construct including:
(i) an expression cassette having:
(one) a first polynucleotide region including a 5′ NCR sequence of an RNA virus and at least an N-terminal portion of a coding sequence of said RNA virus;
(two) a second polynucleotide region including a 3′ UTR sequence of said RNA virus and at least a C-terminal portion of a coding sequence of said virus; and
(three) a third polynucleotide region encoding a reporter molecule, said third polynucleotide region being flanked by said first and said second polynucleotide regions; and
(ii) a promoter sequence being operatively linked to said expression cassette in a manner so as to direct the transcription of a minus strand RNA molecule from said expression cassette when said nucleic acid construct is incubated with said extract, said first and said second polynucleotide regions being selected such that said minus strand RNA molecule transcribed is replicatable by a polymerase of the RNA virus into a plus strand RNA molecule; and
(b) quantifying a level of said reporter molecule to thereby determine the presence of the virus in the cell.

19. The method of claim 18, wherein said reporter molecule is a polypeptide translated from said plus strand RNA molecule.

20. The method of claim 18, further comprising the step of comparing said level of said reporter molecule to that obtained from cells free of the virus.

21. The method of claim 18, wherein at least a portion of said first polynucleotide region is at least 50% identical to a sequence encompassed by nucleotides 1-374 of SEQ ID NO:33.

22. The method of claim 18, wherein at least a portion of said second polynucleotide region is at least 50% identical to a sequence encompassed by nucleotides 9158-9609 of SEQ ID NO:33.

23. A method of detecting the presence of an RNA virus in a cell, the method comprising the steps of:
(a) expressing a nucleic acid construct within the cell, said nucleic acid construct including:
(i) an expression cassette having:
(one) a first polynucleotide region including a 5′ NCR sequence of an RNA virus and at least an N-terminal portion of a coding sequence of said RNA virus;
(two) a second polynucleotide region including a 3′ UTR sequence of said RNA virus and at least a C-terminal portion of a coding sequence of said virus; and
(three)a third polynucleotide region encoding a reporter molecule, said third polynucleotide region being flanked by said first and said second polynucleotide regions; and
(ii) a promoter sequence being operatively linked to said expression cassette in a manner so as to direct the transcription of a minus strand RNA molecule from said expression cassette when said nucleic acid construct is expressed within the cell, said first and said second polynucleotide regions being selected such that said minus strand RNA molecule transcribed is replicatable by a polymerase of the RNA virus into a plus strand RNA molecule; and
(b) quantifying a level of said reporter molecule to thereby determine the presence of the virus in the cell.

24. The method of claim 23, wherein said reporter molecule is a polypeptide translated from said plus strand RNA molecule.

25. The method of claim 23, further comprising the step of comparing said level of said reporter molecule to that obtained from cells free of the virus.

26. The method of claim 23, wherein at least a portion of said first polynucleotide region is at least 50% identical to a sequence encompassed by nucleotides 1-374 of SEQ ID NO:33.

27. The method of claim 23, wherein at least a portion of said second polynucleotide region is at least 50% identical to a sequence encompassed by nucleotides 9158-9609 of SEQ ID NO:33.

28. A method of screening for anti-viral drugs, the method comprising the steps of:
(a) co-incubating a nucleic acid construct, a polynucleotide encoding at least a polymerase of an RNA virus and a potential anti-viral molecule under conditions suitable for transcription and translation of said nucleic acid construct and said polynucleotide encoding at least said polymerase, said nucleic acid construct including:
(i) an expression cassette having:
(one) a first polynucleotide region including a 5′ NCR sequence of an RNA virus and at least an N-terminal portion of a coding sequence of said RNA virus;
(two) a second polynucleotide region including a 3′ UTR sequence of said RNA virus and at least a C-terminal portion of a coding sequence of said virus; and
(three) a third polynucleotide region encoding a reporter molecule, said third polynucleotide region being flanked by said first and said second polynucleotide regions; and
(ii) a promoter sequence being operatively linked to said expression cassette in a manner so as to direct the transcription of a minus strand RNA molecule from said expression cassette when said nucleic acid construct is incubated with said polynucleotide encoding at least said polymerase of said RNA virus under said conditions suitable for transcription and translation, said first and said second polynucleotide regions being selected such that said minus strand RNA molecule transcribed is replicatable by said polymerase of said RNA virus into a plus strand RNA molecule; and
(b) quantifying a level of said reporter molecule to thereby determine the anti-viral activity of said potential anti-viral molecule.

29. The method of claim 28, wherein said reporter molecule is a polypeptide translated from said plus strand RNA molecule.

30. The method of claim 28, further comprising the step of comparing said level of said reporter molecule to that obtained from cells free of the virus.

31. The method of claim 28, wherein said potential anti-viral molecule is selected from the group consisting of a nucleoside or a nucleotide analogue and an immune-modulatory molecule.

32. The method of claim 28, wherein step (a) is effected by introducing said nucleic acid construct, said polynucleotide encoding at least said polymerase of said RNA virus and said potential anti-viral molecule into a cell.

33. The method of claim 28, wherein step (a) is effected by introducing said nucleic acid construct and said potential anti-viral molecule into a cell infected with said RNA virus.

34. The method of claim 28, wherein at least a portion of said first polynucleotide region is at least 50% identical to a sequence encompassed by nucleotides 1-374 of SEQ ID NO:33.

35. The method of claim 28, wherein at least a portion of said second polynucleotide region is at least 50% identical to a sequence encompassed by nucleotides 9158-9609 of SEQ ID NO:33.

36. A method of determining drug resistance of an RNA virus, the method comprising the steps of:
(a) co-incubating a nucleic acid construct, a polynucleotide encoding at least a polymerase of the RNA virus and an anti-viral drug molecule under conditions suitable for transcription and translation of said nucleic acid construct and said polynucleotide encoding at least said polymerase, said nucleic acid construct including:
(i) an expression cassette having:
(one) a first polynucleotide region including a 5′ NCR sequence of an RNA virus and at least an N-terminal portion of a coding sequence of said RNA virus;
(two) a second polynucleotide region including a 3′ UTR sequence of said RNA virus and at least a C-terminal portion of a coding sequence of said virus; and
(three) a third polynucleotide region encoding a reporter molecule, said third polynucleotide region being flanked by said first and said second polynucleotide regions; and
(ii) a promoter sequence being operatively linked to said expression cassette in a manner so as to direct the transcription of a minus strand RNA molecule from said expression cassette when said nucleic acid construct is incubated with said polynucleotide encoding at least said polymerase of the RNA virus under said conditions suitable for transcription and translation, said first and said second polynucleotide regions being selected such that said minus strand RNA molecule transcribed is replicatable by said polymerase of the RNA virus into a plus strand RNA molecule; and
(b) quantifying a level of said reporter molecule to thereby determine the resistance of the RNA virus to said anti-viral drug.

37. The method of claim 36, further comprising the step of comparing said level of said reporter molecule to that obtained from cells free of said anti-viral drug.

38. The method of claim 36, wherein said reporter molecule is a polypeptide translated from said plus strand RNA molecule.

39. The method of claim 36, wherein said anti-viral drug is selected from the group consisting of a nucleoside or nucleotide analog and an immune-modulatory molecule.

40. The method of claim 36, wherein step (a) is effected by introducing said nucleic acid construct, said polynucleotide encoding at least said polymerase of said RNA virus and said anti-viral drug into a cell.

41. The method of claim 36, wherein step (a) is effected by introducing said nucleic acid construct and said anti-viral drug into a cell infected with the RNA virus.

42. The method of claim 36, wherein at least a portion of said first polynucleotide region is at least 50% identical to a sequence encompassed by nucleotides 1-374 of SEQ ID NO:33.

43. The method of claim 36, wherein at least a portion of said second polynucleotide region is at least 50% identical to a sequence encompassed by nucleotides 9158-9609 of SEQ ID NO:33.
